(19) United States
(12) Patent Application Publication
(10) Pub. No.: US 2021/0330251 A1
(43) Pub. Date: Oct. 28, 2021

(54) BRAINWAVE ANALYSIS SYSTEM FOR CORRECTION OF SPINE DISLOCATION, DISPLACEMENT, AND SHIFT AND THE METHOD USING THE SAME

(71) Applicants: Ching Lee, Taipei City (TW); Ruey Yuan Lee, Taipei City (TW)

(72) Inventors: Ching Lee, Taipei City (TW); Ruey Yuan Lee, Taipei City (TW)

(21) Appl. No.: 16/857,200

(22) Filed: Apr. 24, 2020

(51) Int. Cl. A61B 5/00; A61B 5/04; A61B 5/0476

(52) U.S. Cl. CPC A61B 5/4566; A61B 5/04012; A61B 5/0476; A61B 5/0006; A61B 5/7425; A61B 5/6803; A61B 5/7246

(57) ABSTRACT

A spine and ache relation database stores the relations of aches of different portions of human body and the related vertebrae. In the test, the brainwave comparison module compares the brainwaves from the first helmet worn by the tested persons and the second helmet worn by the test apply person so as to determine whether the brainwaves from the first helmet and the second helmet are synchronous. The test applying person contacts the tested person and moreover, the test applying person thinks an aspect of one of the vertebrae continuously and repeatedly in a time period, in that the test apply person may emits voice about the aspect of the vertebrae or only think in the brain silently. The brainwave comparison module compares all the aspects of the selected vertebrae so as to determine which aspects of these vertebrae are displaced or shifted. A method using this system is provided.

### FIELD OF THE INVENTION

The present invention is related to spine treatment by using brainwaves, and in particular to a brainwave analysis system for correction of spine dislocation, displacement, and shift and the method using the same

### BACKGROUND OF THE INVENTION

In many medical research, other than diseases induced from germs, virus, or collisions, all other long term diseases or aches are mainly from subluxation (the dislocation, displacement or shift of vertebraes of spine). The subluxation is mainly from the compression, pulling and displacement of connection nerves so that signal transmission through the nerves is bad and thus the self-treatment ability become worse.

In current medical tests for determination of subluxation is to instruct patients to perform some specific poses so that doctors determine whether vertebraes has displaced by viewing of the doctors. This way is not completely effective, while in current, no automatic system can determine the displacement of vertebrae effectively.

In current medical research, it is believed that transmission of idea between persons is possible. The idea of one person may be transferred from one to another. The transmission of idea is called as induction which is not confined by time or distance. However, one kind of idea transmission can be transferred through contact. This kind of idea transmission is called as contact induction which can be performed by tactile or other human senses. The formation of idea will form variation of brainwaves, and brainwaves can be transferred through contact between people, this is so called contact induction. The contact induction will induce variation of brainwaves from one to another and thus the brainwaves of the two persons are affected by the idea so that the brainwaves of the two persons are varied with specific relations. That is, the brainwaves of two persons are synchronous.

In the prior art, the brainwave detectors are widely used to detect the brainwaves and the brainwaves can be transferred out for many calculations and researches. If the same idea are repeated in one test apply person, the brainwaves of the test apply person will repeat with the repeat of the idea in the rest apply person.

In many researches from the inventor, it is found that when the idea of the test apply person is transferred to a tested person who contacts with the test apply person, then the brainwaves of the tested person will be synchronous with brainwaves of the test apply person. However, from long term researches, it is found that if a vertebrae of the tested person have displacement, dislocation or shift from normal position, and the idea about one specific aspect of this vertebrae from the test apply person can be precisely transferred to the tested person who contacts the test apply person, in this aspect of the vertebrae, the brainwaves of the test applying person and tested person are not synchronously. Thereby, if the synchronization of the two brainwaves can be obtained, this result can be used to determine which aspects of the vertebrae are abnormal.

### SUMMARY OF THE INVENTION

Accordingly, the object of the present invention is to provide a brainwave analysis system for correction of spine dislocation, displacement, and shift, wherein a spine and ache relation database stores the relations of aches of different portions of human body and the related vertebrae. In the test, the brainwave comparison module compares the brainwaves (mainly Alpha waves) from the first helmet worn by the tested persons and the second helmet worn by the test apply person so as to determine whether the brainwaves from the first helmet and the second helmet are synchronous. The test applying person contacts the tested person and moreover, the test applying person thinks an aspect of one of the vertebrae continuously and repeatedly in a time period, in that the test apply person may emits voice about the aspect of the vertebrae or only think in the brain silently. The brainwave comparison module compares all the aspects of the selected vertebrae so as to determine which aspects of these vertebrae are displaced or shifted. In all process is easy and can be performed quickly and precisely. Furthermore the all processes are performed through an automatic system.

To achieve above object, the present invention provides a brainwave analysis system for correction of spine dislocation, displacement, and shift, comprising: a first helmet for detection of brainwaves; the first helmet being worn on a test apply person to apply a test; a second helmet for detection of brainwaves; the second helmet being worn on a tested person which receive tests from the test apply person; each of the first helmet and the second helmet including; a ring having a shape approximately near a round ring; a brainwave detector on the ring for detection of brainwaves; a brainwave chip on the ring for receiving the brainwave values from the brainwave detector; a brainwave transceiver on the ring for transmitting brainwave data which are calculated by the brainwave chip; a processor including a processor transceiver 2lbeing signally connected to the brainwave transceivers of the first helmet and the second helmet; the processor transceiver 21 receiving signals from the brainwave transceivers of the first helmet and the second helmet; the signals being from the test applying person and tested person and being related to a spine test; a spine is formed by a plurality of vertebraes; a spine and ache relation database storing relations of aches of different portions of human body and the related vertebraes; a brainwave comparison module connected to the processor transceiver 21 and the spine and ache relation database; wherein in test, the brainwave comparison module compares the brainwaves from the first helmet worn by the tested persons and the second helmet worn by the test apply person so as to determine whether the brainwaves from the first helmet and the second helmet are synchronous; if in the test, the ratio of synchronous brainwaves and asynchronous brainwaves is over a predetermined number, then a brainwave comparison module outputs that the spine has dislocation, shift or displacement in that aspect; and outputs that the tested person needs to be corrected.

### BRIEF DESCRIPTION OF THE DRAWINGS

FIG. 1 is a schematic view about the first helmet of the present invention.

FIG. 2 is a schematic view about the second helmet of the present invention.

FIG. 3 is an assembled block diagram showing the main components of the present invention.

FIG. 4 shows the process for performing the test of the present invention.

FIG. 5 is a schematic view showing the application of the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

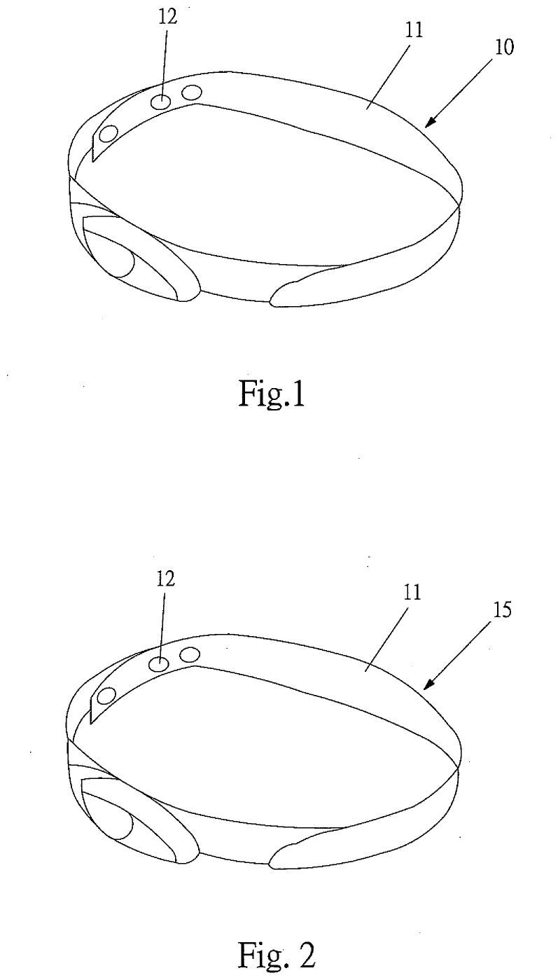

With reference to FIGS. 1 to 5, the structure of the present invention is illustrated. The present invention includes the following elements.

A first helmet 10 (referring to FIG. 1) serves for detection of brainwaves. The first helmet 10 is worn on a test apply person to apply a test.

A second helmet 15 (referring to FIG. 2) serves for detection of brainwaves. The second helmet 15 is worn on a tested person which receive tests from the test apply person.

Each of the first helmet 10 and the second helmet 15 includes the following elements.

A ring 11 has a shape approximately near a round ring.

A brainwave detector 12 is on the ring 11 for detection of brainwaves.

A brainwave chip 13 is on the ring 11 for receiving the brainwave values from the brainwave detector 12. The values including values of Delta brainwaves, Theta. brainwaves, High/Low Alpha brainwaves, High/Low Beta brainwaves, and High/Low Gamma brainwaves.

A brainwave transceiver 14 is on the ring 11 for transmitting brainwave data which are calculated by the brainwave chip 13. The brainwave transceiver 14 is, for example, a blue tooth transceiver.

A processor 20 includes:

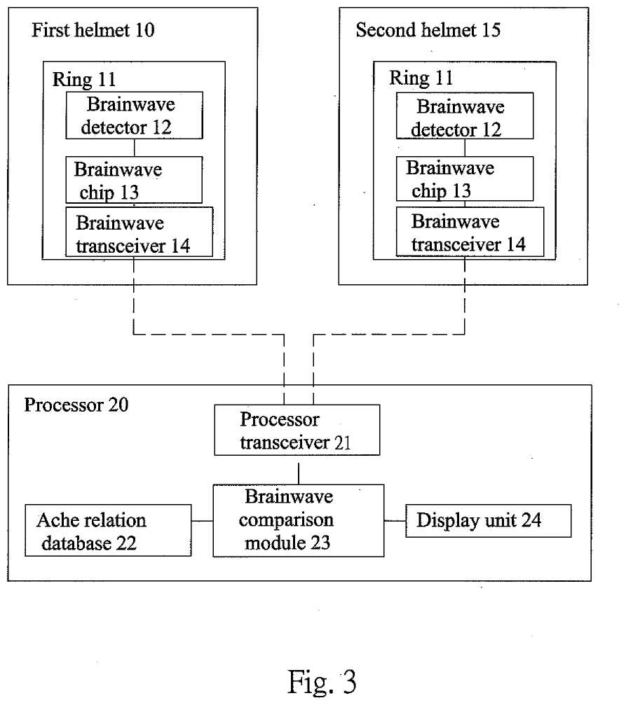

A processor transceiver 21 is signally connected to the brainwave transceivers 14 of the first helmet 10 and the second helmet 15. The processor transceiver 21 receives signals from the brainwave transceivers 14 of the first helmet 10 and the second helmet 15 (referring to FIG. 3). The signals are from the test applying person and tested person and are related to a spine test. A spine is formed by 23 to 24 vertebraes which include 7 cervical vertebraes, 12 Thoracic vertebraes, 5 lumbar vertebraes, etc. Each vertebraes has six aspects of dislocations, displacement, and shift from normal positions, including right and afterward rotations, left and afterward rotation, right displacement, left displacement, front convex movement, and rear convex movement.

A spine and ache relation database 22 stores the relations of aches of different portions of human body and the related vertebraes. For example, the headache is related to 3, 5, and 12 vertebraes of the spine in a spine test. The relations are obtained from a large amount of tests from different persons.

A brainwave comparison module 23 is connected to the processor transceiver 21 and the spine and ache relation database 22. In one test, the brainwave comparison module 23 compares the brainwaves (mainly Alpha waves) from the first helmet 10 worn by the tested persons and the second helmet 15 worn by the test apply person so as to determine whether the brainwaves from the first helmet 10 and the second helmet 15 are synchronous. The synchronization of the brainwaves mean that the ascent and descent of one brainwave are identical to those of another brainwave; and if the ascent and descent of one brainwave are not identical to those of another brainwave, we say that the two waves are not synchronous. If in one test, the ratio of synchronous brainwaves and asynchronous brainwaves is over a predetermined number, then a brainwave comparison module outputs that the spine has dislocation, shift or displacement in that aspect; and a brainwave comparison module outputs that the tested person needs to be corrected.

A display unit 24 is connected to the brainwave comparison module 23 for displacing the comparison result in that test.

The processor 20 may be installed in anyone of various electronic computer devices, such as computers, handsets, tablet computers, PDAs, etc.

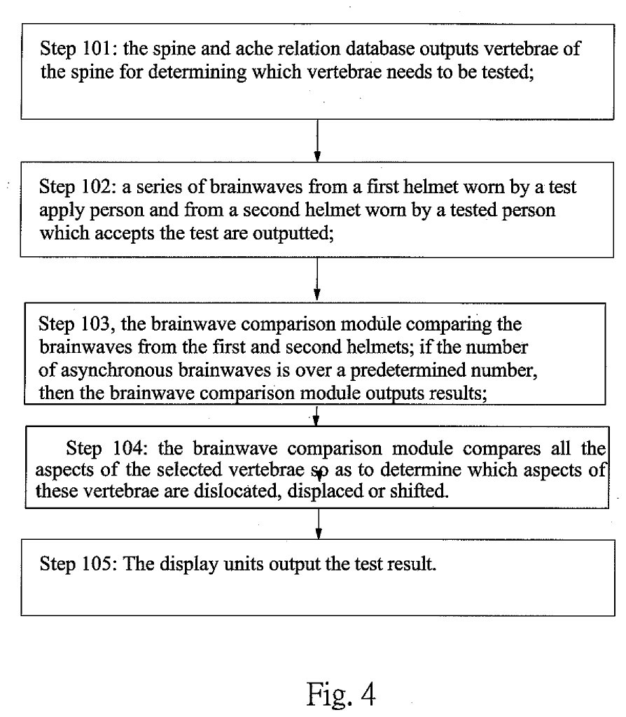

With reference to FIG. 4, the test method by using the brainwave analysis system according to the present invention will be described herein.

Step 101: the spine and ache relation database 22 outputs the vertebrae of the spines according to the ached portion of a tested person needing to be tested so as to determine which vertebrae needs to be tested. For example, if a patient has got headache, the spine and ache relation database 22 outputs that in this state, the 3rd, 5th and 7th vertebraes are necessary to be tested for determining whether these vertebraes have displacements or shift.

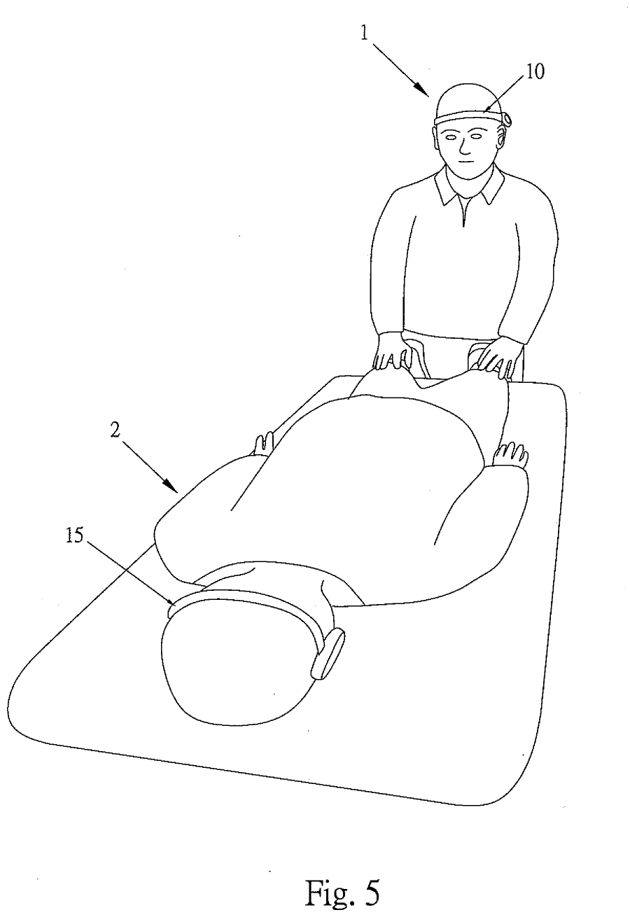

Step 102: A series of brainwaves from the first helmet 10 worn by the test apply person and from the second helmet 15 worn by the tested persons which accepts the test are outputted. In that, the test applying person contacts the tested person and moreover, the test applying person thinks an aspect of one of the vertebrae in his or her brain continuously and repeatedly in a time period, in that the test apply person may emits voice about the aspect of the vertebrae or only think in the brain silently. For example, the hand of the test apply person contacts the leg of the tested person, see FIG. 5. In this period, a series of brainwaves are outputted from the first helmet 10 and the second helmet 15.

Step 103, the brainwave comparison module 23 compares the brainwaves from the first helmet 10 with the brainwaves from the second helmet 15 to determine whether the brainwaves from the first helmet 10 are asynchronous with the brainwaves from the second helmet 15. In that period, there are a plurality of brainwaves outputted from the first helmet 10 and the second helmet 15. Thus the nth brainwave from the first helmet 10 is compared with the 5th brainwaves from the second helmet 15. All the brainwaves generated in the period are compared one by one according to the order of the brainwaves outputted from the first and second helmet. If the number of asynchronous brainwaves is over a predetermined number, then the brainwave comparison module 23 outputs that the vertebrae in that tested aspect have dislocation, displacement or shift from a normal position, and outputs that vertebrae is needed to be corrected in that aspect. The number is determined by many times of tests from many persons in a long time period.

Step 104: The brainwave comparison module 23 compares all the aspects of the selected vertebrae so as to determine which aspects of these vertebrae are dislocated, displaced or shifted.

Step 105: The display units output the test result.

Advantages of the present invention are that: A spine and ache relation database stores the relations of aches of different portions of human body and the related vertebrae. In the test, the brainwave comparison module compares the brainwaves (mainly Alpha waves) from the first helmet worn by the tested persons and the second helmet worn by the test apply person so as to determine whether the brainwaves from the first helmet and the second helmet are synchronous. The test applying person contacts the tested person and moreover, the test applying person thinks an aspect of one of the vertebrae continuously and repeatedly in a time period, in that the test apply person may emits voice about the aspect of the vertebrae or only think in the brain silently. The brainwave comparison module 23 compares all the aspects of the selected vertebrae so as to determine which aspects of these vertebrae are displaced or shifted. In all process is easy and can be performed quickly and precisely. Furthermore the all processes are performed through an automatic system.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A brainwave analysis system for correction of spine dislocation, displacement, and shift, comprising:
a first helmet for detection of brainwaves; the first helmet being worn on a test apply person to apply a test;
a second helmet for detection of brainwaves; the second helmet being worn on a tested person which receive tests from the test apply person;
each of the first helmet and the second helmet including;
a ring having a shape approximately near a round ring;
a brainwave detector on the ring for detection of brainwaves;
a brainwave chip on the ring for receiving the brainwave values from the brainwave detector;
a brainwave transceiver on the ring for transmitting brainwave data which are calculated by the brainwave chip;
a processor including
a processor transceiver 21 being signally connected to the brainwave transceivers of the first helmet and the second helmet; the processor transceiver 21 receiving signals from the brainwave transceivers of the first helmet and the second helmet; the signals being from the test applying person and tested person and being related to a spine test; a spine is formed by a plurality of vertebraes;
a spine and ache relation database storing relations of aches of different portions of human body and the related vertebraes;
a brainwave comparison module connected to the processor transceiver 21 and the spine and ache relation database; wherein in test, the brainwave comparison module compares the brainwaves from the first helmet worn by the tested persons and the second helmet worn by the test apply person so as to determine whether the brainwaves from the first helmet and the second helmet are synchronous; if in the test, the ratio of synchronous brainwaves and asynchronous brainwaves is over a predetermined number, then a brainwave comparison module outputs that the spine has dislocation, shift or displacement in that aspect; and outputs that the tested person needs to be corrected.

2. The brainwave analysis system for correction of spine dislocation as claimed in claim 1, wherein the synchronization of the brainwaves mean that the ascent and descent of one brainwave are identical to those of another brainwave; and if the ascent and descent of one brainwave are not identical to those of another brainwave, we say that the two waves are asynchronous.

3. The brainwave analysis system for correction of spine dislocation as claimed in claim 1, further comprising a display unit connected to the brainwave comparison module for displacing the comparison result in that test.

4. The brainwave analysis system for correction of spine dislocation as claimed in claim 1, wherein each vertebraes has six aspects of dislocations, displacement, and shift from normal positions, including right and afterward rotations, left and afterward rotation, right displacement, left displacement, front convex movement, and rear convex movement.

5. The brainwave analysis system for correction of spine dislocation as claimed in claim 1, wherein the brainwaves for comparison are Alpha waves.

6. The brainwave analysis system for correction of spine dislocation as claimed in claim 1, wherein the processor is installed in anyone of various electronic computer devices including computers, handsets, tablet computers, PDAs, etc.

7. A test method using the system of claim 1, the method comprising the steps of:
Step 101: the spine and ache relation database outputs vertebrae of the spine according to an ached portion of a tested person needing to be tested so as to determine which vertebrae needs to be tested;
Step 102: a series of brainwaves from a first helmet worn by a test apply person and from a second helmet worn by a tested person which accepts the test are outputted; in that, the test applying person contacts the tested person and moreover, the test applying person thinks an aspect of one of the vertebrae in his or her brain continuously and repeatedly in a time period, in that the test apply person emits voice about the aspect of the vertebrae or only think in the brain silently; and in this period, a series of brainwaves are outputted from the first helmet and the second helmet.
Step 103, the brainwave comparison module comparing the brainwaves from the first helmet with the brainwaves from the second helmet to determine whether the brainwaves from the first helmet are asynchronous with the brainwaves from the second helmet, in that period, there are a plurality of brainwaves outputted from the first helmet and the second helmet; all the brainwaves generated in the period are compared one by one according to orders of the brainwaves outputted from the first helmet and second helmet; if the number of asynchronous brainwaves is over a predetermined number, then the brainwave comparison module outputs that the vertebrae in that tested aspect have dislocation, displacement or shift from a normal position and outputs vertebrae is needed to be corrected in that aspect;
Step 104: the brainwave comparison module compares all the aspects of the selected vertebrae so as to determine which aspects of these vertebrae are dislocated, displaced or shifted.

8. The method as claimed in claim 7, wherein the number is determined by many times of tests from many persons in a long time period.
